(19) United States
(12) Patent Application Publication
(10) Pub. No.: US 2026/0131114 A1
(43) Pub. Date: May 14, 2026

(54) BI-DIRECTIONAL STEERABLE CATHETER

(71) Applicant: BOSTON SCIENTIFIC SCIMED, INC., MAPLE GROVE, MN (US)

(72) Inventors: Nicholas Lee Tassoni, Andover, MN (US); Jan-Hung Chen, St. Paul, MN (US); Christopher J Clark, St. Michael, MN (US); Ryan Robert Davis, Plymouth, MN (US)

(73) Assignee: BOSTON SCIENTIFIC SCIMED, INC., MAPLE GROVE, MN (US)

(21) Appl. No.: 19/332,526

(22) Filed: Sep. 18, 2025

Related U.S. Application Data
(60) Provisional application No. 63/696,027, filed on Sep. 18, 2024.

(51) Int. Cl. A61M 25/01

(52) U.S. Cl. CPC A61M 25/0136; A61M 25/0147; A61M 2025/015

(57) ABSTRACT

A steering mechanism includes an axial translation mechanism including an elongate shaft having first and second helical threads extending along the elongate shaft, a first carriage member operatively engaged with the first helical thread, and a second carriage member operatively engaged with the second helical thread. Rotation of the elongate shaft translates the first and second carriage members in opposite directions. A bi-directional steerable catheter includes a handle including the steering mechanism, and an elongate sheath extending distally from the handle. The axial translation mechanism is coupled to first and second steering wires extending within the elongate sheath. The first carriage member is coupled to the first steering wire, and the second carriage member is coupled to the second steering wire. The first helical thread may axially overlap the second helical thread. The elongate shaft may include clockwise discontinuous threading overlaid with counterclockwise discontinuous threading.

### CROSS-REFERENCE TO RELATED APPLICATIONS

This application claims the benefit of priority of U.S. Provisional Application No. 63/696,027 filed Sep. 18, 2024, the entire disclosure of which is hereby incorporated by reference.

### TECHNICAL FIELD

The disclosure relates generally to medical devices and more particularly to mechanisms for steering catheters, sheaths, and/or elongate tubular shafts.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

In one example, a steering mechanism for a bi-directional steerable catheter may comprise an axial translation mechanism disposable within a handle of the bi-directional steerable catheter. The axial translation mechanism may comprise an elongate shaft having a circumference and comprising a first helical thread extending along a length of the elongate shaft in a first helical direction, and a second helical thread extending along the length of the elongate shaft in a second helical direction different from the first helical direction, a first carriage member couplable to a first steering wire and operatively engaged with the first helical thread, and a second carriage member couplable to a second steering wire and operatively engaged with the second helical thread. Rotation of the elongate shaft may be configured to translate the first carriage member and the second carriage member in opposite directions along the elongate shaft.

In addition, or alternatively, to any example disclosed herein, the first carriage member extends circumferentially around at least a portion of the circumference of the elongate shaft.

In addition, or alternatively, to any example disclosed herein, the second carriage member extends circumferentially around at least a portion of the circumference of the elongate shaft.

In addition, or alternatively, to any example disclosed herein, the first carriage member and the second carriage member are configured to axially pass by each other as they translate along the elongate shaft.

In addition, or alternatively, to any example disclosed herein, the first carriage member and the second carriage member are disposed coaxial with the elongate shaft.

In addition, or alternatively, to any example disclosed herein, the steering mechanism may comprise a knob configured to rotate relative to the handle.

In addition, or alternatively, to any example disclosed herein, the knob is fixedly attached to the elongate shaft.

In addition, or alternatively, to any example disclosed herein, the knob is axially spaced apart from the first helical thread and the second helical thread.

In addition, or alternatively, to any example disclosed herein, the axial translation mechanism is configured to shift an elongate sheath extending distally from the handle between a relaxed configuration, a deflected configuration, and a straightened configuration.

In addition, or alternatively, to any example disclosed herein, the steering mechanism may comprise a first steering wire configured to extend through the elongate sheath from the first carriage member to a distal pull ring disposed adjacent a distal tip of the elongate sheath, and a second steering wire configured to extend through the elongate sheath from the second carriage member to the distal pull ring.

In addition, or alternatively, to any example disclosed herein, actuation of the axial translation mechanism is configured to selectively apply tension to the first steering wire and the second steering wire to shift the elongate sheath between the relaxed configuration, the deflected configuration, and the straightened configuration.

In addition, or alternatively, to any example disclosed herein, and in another example, a bi-directional steerable catheter may comprise a handle comprising a steering mechanism and an elongate sheath extending distally from the handle, wherein the steering mechanism comprises an axial translation mechanism disposed within the handle, the axial translation mechanism being coupled to a first steering wire extending within the elongate sheath and a second steering wire extending within the elongate sheath.

In addition, or alternatively, to any example disclosed herein, the axial translation mechanism may comprise an elongate shaft having a circumference and comprising a first helical thread extending along an exterior of the elongate shaft in a first helical direction, and a second helical thread extending along the exterior of the elongate shaft in a second helical direction different from the first helical direction, wherein the first helical thread axially overlaps the second helical thread, a first carriage member operatively engaged with the first helical thread, the first carriage member being coupled to the first steering wire, and a second carriage member operatively engaged with the second helical thread, the second carriage member being coupled to the second steering wire. Rotation of the elongate shaft may be configured to translate the first carriage member and the second carriage member in opposite directions along the elongate shaft.

In addition, or alternatively, to any example disclosed herein, the first helical thread intersects the second helical thread.

In addition, or alternatively, to any example disclosed herein, the first helical thread is discontinuous along the elongate shaft.

In addition, or alternatively, to any example disclosed herein, the second helical thread is discontinuous along the elongate shaft.

In addition, or alternatively, to any example disclosed herein, the bi-directional steerable catheter may comprise a rotatable knob disposed outside of the handle, wherein the rotatable knob is fixedly attached to the elongate shaft.

In addition, or alternatively, to any example disclosed herein, the rotatable knob is monolithically formed with the elongate shaft.

In addition, or alternatively, to any example disclosed herein, and in another example, a bi-directional steerable catheter may comprise a handle comprising a steering mechanism, and an elongate sheath extending distally from the handle, wherein the steering mechanism comprises an axial translation mechanism coupled to a first steering wire extending within the elongate sheath and a second steering wire extending within the elongate sheath.

In addition, or alternatively, to any example disclosed herein, the axial translation mechanism may comprise an elongate shaft having a threaded portion comprising clockwise discontinuous threading overlaid with counterclockwise discontinuous threading, a first carriage member operatively engaged with the threaded portion, the first carriage member being coupled to the first steering wire, and a second carriage member operatively engaged with the threaded portion, the second carriage member being coupled to the second steering wire. Rotation of the elongate shaft may be configured to translate the first carriage member and the second carriage member in opposite directions along the elongate shaft.

In addition, or alternatively, to any example disclosed herein, the clockwise discontinuous threading and the counterclockwise discontinuous threading are formed by intersecting helical grooves extending radially inward from an outermost extent of the threaded portion.

In addition, or alternatively, to any example disclosed herein, the intersecting helical grooves form a plurality of diamond-shaped projections extending radially outward from an innermost extent of the intersecting helical grooves.

In addition, or alternatively, to any example disclosed herein, each diamond-shaped projection of the plurality of diamond-shaped projections forms a portion of the clockwise discontinuous threading and a portion of the counterclockwise discontinuous threading.

In addition, or alternatively, to any example disclosed herein, the first carriage member is operatively engaged with the clockwise discontinuous threading and the second carriage member is operatively engaged with the counterclockwise discontinuous threading.

The above summary of some embodiments, aspects, and/or examples is not intended to describe each disclosed embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of an example steerable catheter;

FIG. 2 illustrates selected aspects of the example steerable catheter of FIG. 1;

FIG. 3 is a cross-sectional view taken along line 3-3 of FIG. 2 illustrating selected aspects of the example steerable catheter of FIGS. 1-2;

FIG. 4 illustrates selected aspects of the example steerable catheter of FIG. 1;

FIG. 4A is a cross-sectional view of FIG. 4 taken along line 4A-4A;

FIGS. 5-6 illustrate selected aspects of steering the example steerable catheter of FIG. 1 in a first direction;

FIGS. 7-8 illustrate selected aspects of steering the example steerable catheter of FIG. 1 in a second direction;

FIG. 9 illustrates selected aspects of an elongate shaft of an example steerable catheter according to the disclosure;

FIG. 9A is a detailed view illustrating selected aspects of a portion of the elongate shaft of FIG. 9; and

FIGS. 10-13 illustrate selected aspects of an alternative configuration of an example steerable catheter according to the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

In some medical procedures, delivery and/or access sheaths may be routed percutaneously into a body cavity, lumen, and/or treatment site. Navigation through patient vasculature and/or organs may include steering through tortuous anatomy and/or directing a distal end of the delivery and/or access sheath into a body cavity, lumen, and/or treatment site. Examples of medical devices suitable for use in medical procedures, such as but not limited to left atrial appendage closure, aortic valve replacement, mitral valve replacement, septal defect repair, etc., are described herein. Existing medical devices may have certain advantages and/or disadvantages. There is an ongoing need for alternative steerable medical devices for delivering medical implants and/or conducting other treatment procedures.

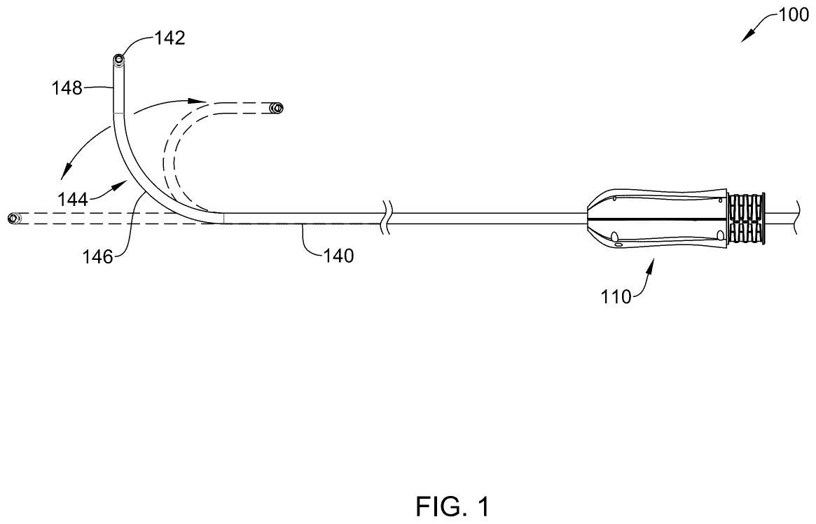

FIG. 1 illustrates selected aspects of a bi-directional steerable catheter 100. In some embodiments, the bi-directional steerable catheter 100 may be any one of a variety of catheters, such as an intravascular catheter. Examples of intravascular catheters may include, but are not limited to, balloon catheters, atherectomy catheters, device delivery catheters, drug delivery catheters, diagnostic catheters, and guide catheters. In some embodiments, the bi-directional steerable catheter 100 may take the form of other suitable guiding, diagnosing, or treating devices (including endoscopic instruments, laparoscopic instruments, etc., and the like) and it may be suitable for use at various locations and/or body lumens within a patient.

The bi-directional steerable catheter 100 may comprise a handle 110 and an elongate sheath 140 extending distally from the handle 110. In some embodiments, the bi-directional steerable catheter 100, the handle 110, and/or the elongate sheath 140 may include a guidewire port, a side port, a fluid flush port, an imaging access port, or other suitable ports, access points, or functional features.

In some embodiments, the elongate sheath 140 may extend into and/or through the handle 110. In some embodiments, the elongate sheath 140 may extend into and/or through a distal opening in the handle housing. In some embodiments, the elongate sheath 140 may be fixedly attached to the handle housing. In some embodiments, a proximal portion of the elongate sheath 140 may include a key element configured to non-rotatably engage one or more lock elements fixedly attached to an inner surface of the handle housing. In some embodiments, the key element may be bonded to an outer surface of the elongate sheath 140. In some embodiments, the key element may be monolithically formed with the elongate sheath 140. In some embodiments, the key element may be welded (e.g., heat weld, sonic weld, vibration weld, etc.) to the elongate sheath 140. In some embodiments, the key element may be melted together (e.g., reflowed) with the elongate sheath 140 such that material of the key element is co-mingled with material of the elongate sheath 140 at a molecular level. In some embodiments, the handle housing may include one or more lock elements fixedly attached to and/or monolithically formed with the inner surface of the handle housing. In some embodiments, the one or more lock elements may be formed as ribs or other structural support members configured to increase the rigidity of the handle housing and permit torque transfer between the handle housing and the elongate sheath 140. In some embodiments, the elongate sheath 140 may have a relaxed configuration. The elongate sheath 140 is shown in the relaxed configuration in solid lines in FIG. 1. In some embodiments, the elongate sheath 140 may be self-biased toward, and/or in the absence of any outside forces may return to, the relaxed configuration. In some embodiments, the elongate sheath 140 may be configured to remain in a given configuration in the absence of force and/or torque applied thereto (e.g., the elongate sheath 140 is not self-biased toward any particular configuration).

In some embodiments, the elongate sheath 140 may include an atraumatic distal tip 142. In some embodiments, the elongate sheath 140 may include a distal portion 144 having a first curve 146 and a second curve 148, such that the elongate sheath 140 has a preset double curve, in the relaxed configuration, as shown in FIG. 1. In some embodiments, the first curve 146 may be preset to curve upwards, as viewed from the side. Other configurations are also contemplated. In some embodiments, the second curve 148 may be preset to curve to the left, as viewed proximally to distally along the elongate sheath 140. Other configurations are also contemplated.

In some embodiments, the distal portion 144 and/or the first curve 146 may be configured to bend or deflect in a first direction, wherein the atraumatic distal tip 142 is bent and/or moved towards and/or closer to the handle 110, toward and/or to a deflected configuration, as shown in phantom in FIG. 1. In some embodiments, the distal portion 144 and/or the first curve 146 may be configured to bend or deflect in a second direction opposite the first direction, wherein the atraumatic distal tip 142 is bent and/or moved away from and/or farther from the handle 110, toward and/or to a straightened configuration, as shown in phantom in FIG. 1. In some embodiments, the elongate sheath 140 may have only a single curve in the normal or relaxed configuration. In some embodiments, the elongate sheath 140 may be substantially straight in the normal or relaxed configuration. Other configurations, including combinations of those described herein, are also contemplated.

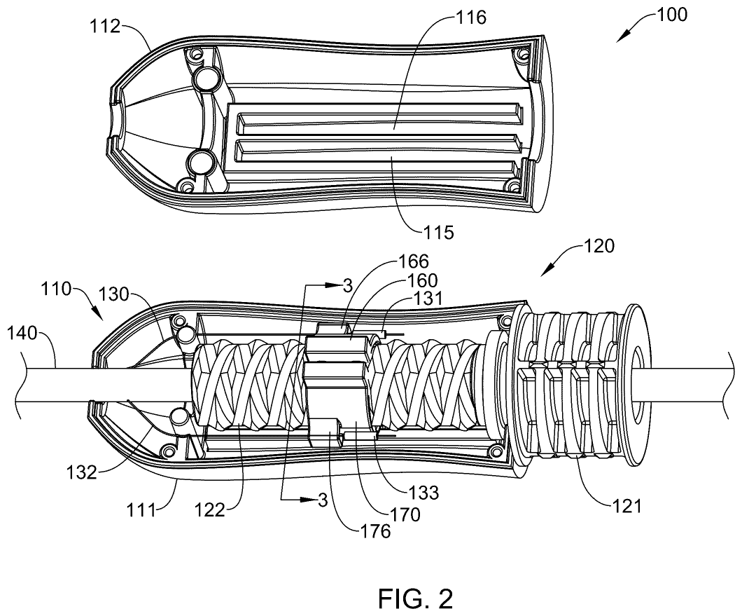

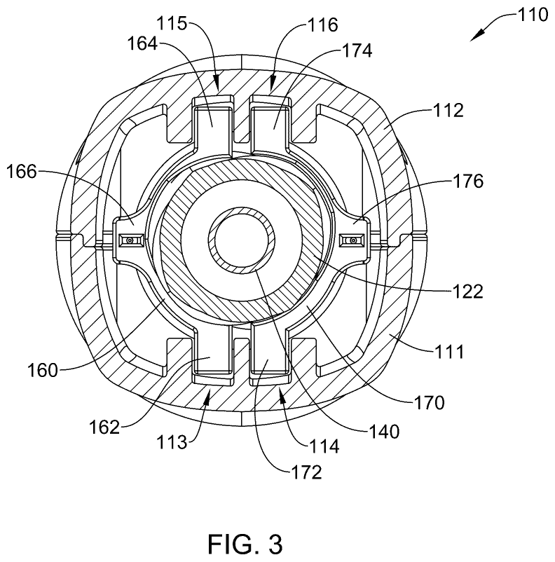

FIGS. 2 and 3 illustrate selected features of the bi-directional steerable catheter 100. The handle 110 may include a handle housing. The handle housing may comprise a first handle shell 111 (e.g., a right handle shell, a bottom handle shell, etc.) and a second handle shell 112 (e.g., a left handle shell, a top handle shell, etc.). In some embodiments, the handle housing may form a clam shell configuration. In some embodiments, the first handle shell 111 and the second handle shell 112 may be secured together, using fasteners, a snap fit, an interference fit, etc. Some suitable but non-limiting materials for the handle 110, the handle housing, the first handle shell 111, the second handle shell 112, etc., including but not limited to polymeric materials, metallic materials, and/or composite materials, are described below. In some embodiments, the handle 110, the handle housing, the first handle shell 111, the second handle shell 112 may preferably be formed from a polymeric material. In one non-limiting example, the handle 110, the handle housing, the first handle shell 111, the second handle shell 112 may preferably be formed from acrylonitrile butadiene styrene (ABS). Other configurations and/or materials are also contemplated. In the view shown in FIG. 2, the second handle shell 112 of the handle housing has been flipped over to show some internal components of the handle 110. The cross-sectional view of FIG. 3, taken along the line 3-3 of FIG. 2, is shown with the second handle shell 112 of the handle housing in place (e.g., as seen in FIG. 1) to illustrate relative positioning of and/or interaction between selected features.

In some embodiments, the bi-directional steerable catheter 100 may comprise a steering mechanism comprising an axial translation mechanism 120 disposable within and/or disposed within the handle 110 of the bi-directional steerable catheter 100. In some embodiments, the axial translation mechanism 120 may comprise an elongate shaft 122 having a circumference. In some embodiments, the elongate shaft 122 may have a threaded portion. In some embodiments, the elongate shaft 122 may be rotatably disposable within and/or rotatably disposed within the handle housing. In some embodiments, the elongate shaft 122 may be rotatable relative to the handle housing.

In some embodiments, the elongate sheath 140 may extend within and/or through the elongate shaft 122. In some embodiments, the elongate sheath 140 may be disposed coaxial with the elongate shaft 122. In some embodiments, the elongate shaft 122 may be configured to rotate around and/or relative to the elongate sheath 140.

In some embodiments, the steering mechanism may comprise a rotatable knob 121 configured to rotate relative to the handle 110 and/or the handle housing. In some embodiments, the rotatable knob 121 may be fixedly attached to the elongate shaft 122. In some embodiments, the rotatable knob 121 may be monolithically formed with the elongate shaft 122. In some embodiments, the rotatable knob 121 may be configured to rotate around and/or relative to the elongate sheath 140. In some embodiments, rotation of the rotatable knob 121 relative to the handle 110 and/or the handle housing causes rotation of the elongate shaft 122 relative to the handle 110 and/or the handle housing. Some suitable but non-limiting materials for the elongate shaft 122 and/or the rotatable knob 121, including but not limited to polymeric materials, metallic materials, and/or composite materials, are described below. In some embodiments, the elongate shaft 122 and/or the rotatable knob 121 may preferably be formed from a polymeric material. In one non-limiting example, the elongate shaft 122 and/or the rotatable knob 121 may preferably be formed from acrylonitrile butadiene styrene (ABS). Other configurations and/or materials are also contemplated.

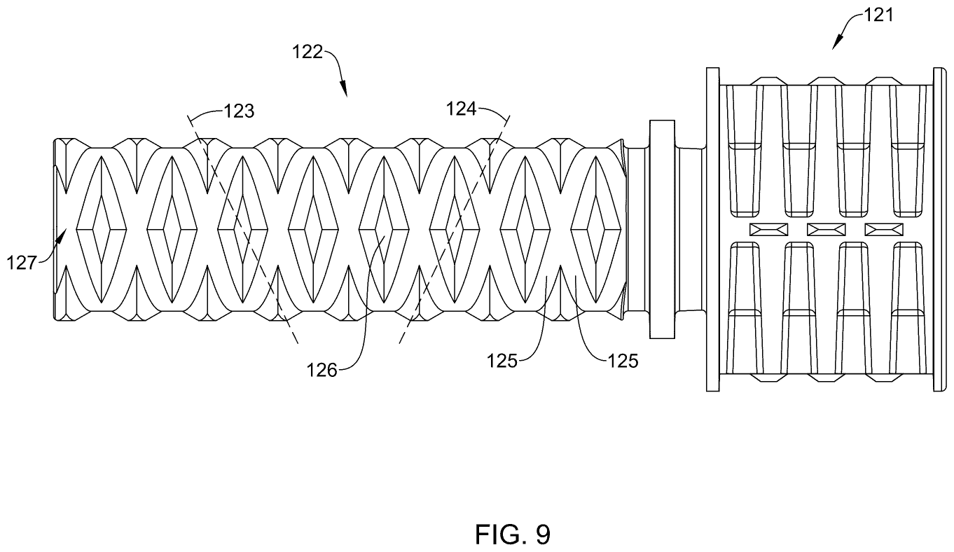

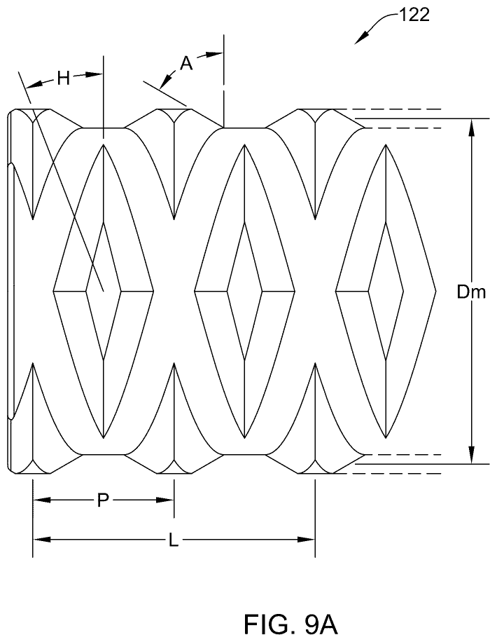

Turning briefly to FIG. 9, selected aspects of the elongate shaft 122 and/or the rotatable knob 121 are described in greater detail. In some embodiments, the elongate shaft 122 and/or the threaded portion may comprise a first helical thread 123 extending along a length of the elongate shaft 122 in a first helical direction. In some embodiments, the elongate shaft 122 and/or the threaded portion may comprise a second helical thread 124 extending along the length of the elongate shaft 122 in a second helical direction different from the first helical direction. In some embodiments, the first helical thread 123 may extend along an exterior of the elongate shaft 122 and/or the threaded portion in the first helical direction. In some embodiments, the second helical thread 124 may extend along the exterior of the elongate shaft 122 and/or the threaded portion in the second helical direction. In some embodiments, the first helical thread 123 may axially overlap the second helical thread 124. In some embodiments, the first helical thread 123 may intersect the second helical thread 124. In some embodiments, the first helical thread 123 may be discontinuous along the elongate shaft 122 and/or the threaded portion. In some embodiments, the second helical thread 124 may be discontinuous along the elongate shaft 122 and/or the threaded portion. In some embodiments, the rotatable knob 121 may be axially spaced apart from and/or the threaded portion. In some embodiments, the rotatable knob 121 may be axially spaced apart from the first helical thread 123 and the second helical thread 124.

In some embodiments, the threaded portion of the elongate shaft 122 may comprise clockwise discontinuous threading (e.g., the first helical thread 123) extending in a proximal to distal direction overlaid with counterclockwise discontinuous threading (e.g., the second helical thread 124) extending in the proximal to distal direction. In some embodiments, the clockwise discontinuous threading (e.g., the first helical thread 123) and the counterclockwise discontinuous threading (e.g., the second helical thread 124) are formed by intersecting helical grooves 125 extending radially inward from an outermost extent of the elongate shaft 122 and/or the threaded portion. In some embodiments, the intersecting helical grooves 125 may form a plurality of diamond-shaped projections 126 extending radially outward from an innermost radial extent of the intersecting helical grooves 125. In some embodiments, each diamond-shaped projection of the plurality of diamond-shaped projections 126 forms a portion of the clockwise discontinuous threading (e.g., the first helical thread 123) and a portion of the counterclockwise discontinuous threading (e.g., the second helical thread 124).

In some embodiments, the threaded portion of the elongate shaft 122 may include single-start threads. In some embodiments, the threaded portion of the elongate shaft 122 may include multi-start threads. Multi-start threads may include two or more parallel threads on the same component (e.g., the elongate shaft 122), which may allow for more linear movement (of the carriage members described herein) per revolution of the elongate shaft 122. In some embodiments, multi-start threads may increase surface contact between adjacent and/or interacting elements for load transfer. In the example configuration shown in FIG. 9, the threaded portion of the elongate shaft 122 includes double-start threads for the first helical thread 123 and the second helical thread 124. A first thread start 127 is shown in FIG. 9, while a second thread start is not visible. The second thread start in the example configuration shown in FIG. 9 is disposed on the opposite side of the elongate shaft 122 (e.g., about 180 degrees around the circumference of the elongate shaft 122) from the first thread start 127. Other configurations of multi-start threads (e.g., three-start threads, four-start threads, etc.) are also contemplated.

Returning to FIGS. 2 and 3, in some embodiments, the axial translation mechanism 120 may comprise a first carriage member 160 couplable to a first steering wire 130. In some embodiments, the steering mechanism may comprise the axial translation mechanism 120 coupled to the first steering wire 130. In some embodiments, the first carriage member 160 may be coupled to the first steering wire 130. In some embodiments, the first carriage member 160 may be operatively engaged with the elongate shaft 122 and/or the threaded portion of the elongate shaft 122. In some embodiments, the first carriage member 160 may be operatively engaged with the first helical thread 123 and/or the clockwise discontinuous threading. In some embodiments, the first steering wire 130 may be configured to extend and/or may extend within and/or through the elongate sheath 140.

In some embodiments, the axial translation mechanism 120 may comprise a second carriage member 170 couplable to a second steering wire 132. In some embodiments, the steering mechanism may comprise the axial translation mechanism 120 coupled to the second steering wire 132. In some embodiments, the second carriage member 170 may be coupled to the second steering wire 132. In some embodiments, the second carriage member 170 may be operatively engaged with the elongate shaft 122 and/or the threaded portion of the elongate shaft 122. In some embodiments, the second carriage member 170 may be operatively engaged with the second helical thread 124 and/or the counterclockwise discontinuous threading. In some embodiments, the second steering wire 132 may be configured to extend and/or may extend within and/or through the elongate sheath 140.

In some embodiments, the handle housing may comprise one or more slide features configured to guide and/or constrain the first carriage member 160 and the second carriage member 170. In some embodiments, the one or more slide features may permit axial sliding movement of the first carriage member 160 and the second carriage member 170 within the handle housing. In some embodiments, the one or more slide features may limit and/or constrain the first carriage member 160 and the second carriage member 170 to axial sliding movement within the handle housing. In some embodiments, the first handle shell 111 may comprise a first guide slot 113 extending along an inner surface of the first handle shell 111 and configured to engage with and/or receive the first carriage member 160, and a second guide slot 114 extending along the inner surface of the first handle shell 111 and configured to engage with and/or receive the second carriage member 170. In some embodiments, the second handle shell 112 may comprise a first guide slot 115 extending along an inner surface of the second handle shell 112 and configured to engage with and/or receive the first carriage member 160, and a second guide slot 116 extending along the inner surface of the second handle shell 112 and configured to engage with and/or receive the second carriage member 170, as seen in FIG. 3.

In some embodiments, the first guide slot 113 of the first handle shell 111 may be disposed opposite the first guide slot 115 of the second handle shell 112 with respect to a plane containing a central longitudinal axis of the handle 110 and/or a central longitudinal axis of the elongate shaft 122. In some embodiments, the second guide slot 114 of the first handle shell 111 may be disposed opposite the second guide slot 116 of the second handle shell 112 with respect to the plane containing the central longitudinal axis of the handle 110 and/or the central longitudinal axis of the elongate shaft 122.

In some embodiments, the first carriage member 160 may comprise a first slide extension 162 and a second slide extension 164, as seen in FIG. 3. The first slide extension 162 may be configured to slidably engage and/or may be slidably engaged with the first guide slot 113 of the first handle shell 111. The second slide extension 164 may be configured to slidably engage and/or may be slidably engaged with the first guide slot 115 of the second handle shell 112. In some embodiments, the first carriage member 160 may extend circumferentially around at least a portion of the circumference of the elongate shaft 122. In some embodiments, the first carriage member 160 may extend circumferentially around less than half of the circumference of the elongate shaft 122. In some embodiments, the first carriage member 160 may comprise a concave shape facing toward the elongate shaft 122. In some embodiments, the first carriage member 160 may comprise a first ear 166 extending radially outward from the elongate shaft 122 and/or extending away from the concave shape of the first carriage member 160 facing toward the elongate shaft 122. The first ear 166 may be configured to receive and/or engage with the first steering wire 130. In some embodiments, the first steering wire 130 may pass through an aperture formed in the first ear 166. In some embodiments, the first steering wire 130 may comprise a first wire lug 131 (e.g., FIG. 2) disposed proximal of the first ear 166, wherein the first wire lug 131 is configured to engage with the first ear 166 and is unable to pass through the aperture formed in the first ear 166. In some embodiments, the first wire lug 131 may be configured to couple the first steering wire 130 to the first carriage member 160 and/or the first ear 166.

In some embodiments, the second carriage member 170 may comprise a first slide extension 172 and a second slide extension 174, as seen in FIG. 3. The first slide extension 172 may be configured to slidably engage and/or may be slidably engaged with the second guide slot 114 of the first handle shell 111. The second slide extension 174 may be configured to slidably engage and/or may be slidably engaged with the second guide slot 116 of the second handle shell 112. In some embodiments, the second carriage member 170 may extend circumferentially around at least a portion of the circumference of the elongate shaft 122. In some embodiments, the second carriage member 170 may extend circumferentially around less than half of the circumference of the elongate shaft 122. In some embodiments, the second carriage member 170 may comprise a concave shape facing toward the elongate shaft 122. In some embodiments, the second carriage member 170 may comprise a second ear 176 extending radially outward from the elongate shaft 122 and/or extending away from the concave shape of the second carriage member 170 facing toward the elongate shaft 122. The second ear 176 may be configured to receive and/or engage with the second steering wire 132. In some embodiments, the second steering wire 132 may pass through an aperture formed in the second ear 176. In some embodiments, the second steering wire 132 may comprise a second wire lug 133 (e.g., FIG. 2) disposed proximal of the second ear 176, wherein the second wire lug 133 is configured to engage with the second ear 176 and is unable to pass through the aperture formed in the second ear 176. In some embodiments, the second wire lug 133 may be configured to couple the second steering wire 132 to the second carriage member 170 and/or the second ear 176.

In some embodiments, the first carriage member 160 and/or the second carriage member 170 may be formed from a polymeric material. In one non-limiting example, the first carriage member 160 and/or the second carriage member 170 may be formed from polyoxymethylene homopolymer (POM-H; for example, DELRIN®). Some other examples of polymeric materials that may be suitable for use with the first carriage member 160 and/or the second carriage member 170 are discussed below.

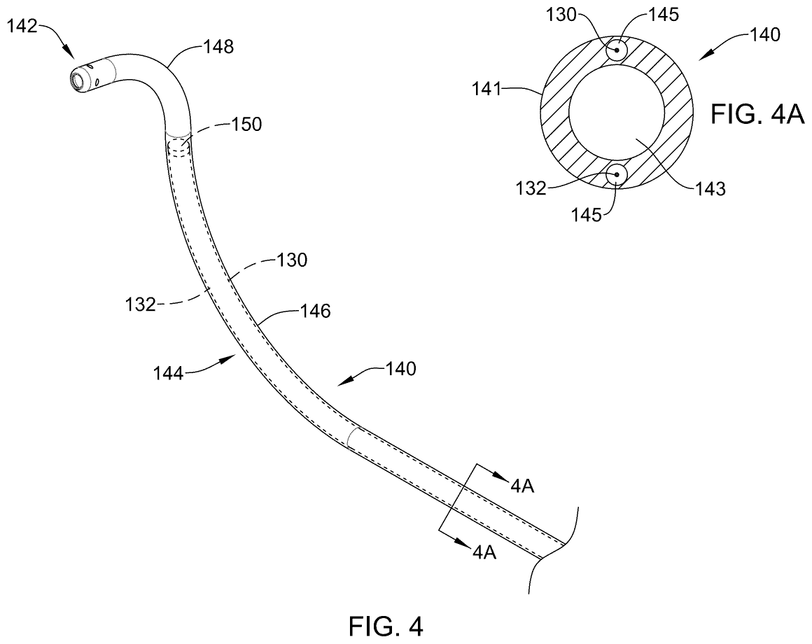

Turning now to FIGS. 4 and 4A, which should be viewed in combination with FIGS. 1 and 2, the elongate sheath 140 may include a wall 141 defining a central lumen 143 extending from a proximal end to the soft and/or atraumatic distal tip 142 along the central longitudinal axis of the elongate sheath 140. In some embodiments, the central lumen 143 may be coaxial with the central longitudinal axis of the elongate sheath 140. In some embodiments, the central lumen 143 may be a guidewire lumen. In some embodiments, the central lumen 143 may be a device lumen used to deliver a medical device or implant. In some embodiments, the central lumen 143 may have multiple uses. The elongate sheath 140 may include a plurality of steering wire lumens 145 extending and/or disposed within the wall 141. In some embodiments, the plurality of steering wire lumens 145 may include a first steering wire lumen and a second steering wire lumen. In some embodiments, the plurality of steering wire lumens 145 may include more than two steering wire lumens. In some embodiments, the plurality of steering wire lumens 145 may be oriented substantially parallel to the central lumen 143 and/or the central longitudinal axis of the elongate sheath 140. In some embodiments, the plurality of steering wire lumens 145 may be disposed opposite each other and/or on opposite sides of the elongate sheath 140 relative to the central lumen 143 and/or the central longitudinal axis of the elongate sheath 140. Other configurations are also contemplated.

In some embodiments, the plurality of steering wire lumens 145 may have a circular cross-sectional shape, as shown in FIG. 4A. However, the cross-sectional shape shown in FIG. 4A is merely exemplary and is not intended to be limiting. In some embodiments, the plurality of steering wire lumens 145 may have other cross-sectional shapes. For example, in some embodiments, the plurality of steering wire lumens 145 may have a rectangular cross-sectional shape, an ovoid cross-sectional shape, a square cross-sectional shape, a polygonal cross-sectional shape, etc. In some embodiments, the plurality of steering wire lumens 145 may have a cross-sectional shape that is regular and/or symmetrical. In some embodiments, the plurality of steering wire lumens 145 may have a cross-sectional shape that is irregular and/or asymmetrical. Other configurations are also contemplated.

As discussed herein, a distal pull ring 150 may be disposed within the distal portion 144 of the elongate sheath 140. In some embodiments, the distal pull ring 150 may be disposed proximal to the second curve 148 and/or the atraumatic distal tip 142. In some embodiments, the distal pull ring 150 may be disposed proximate a distal end of the first curve 146. In some embodiments, the distal pull ring 150 may be embedded within the wall 141 of the elongate sheath 140. In some embodiments, the distal pull ring 150 may be secured, bonded, and/or fixedly attached to an inner surface of the wall 141 of the elongate sheath 140. Other configurations are also contemplated. Some suitable but non-limiting materials for the distal pull ring 150 are described below.

The first steering wire 130 and the second steering wire 132 may each be slidably disposed within the plurality of steering wire lumens 145. In one example, the first steering wire 130 may be slidably disposed within the first steering wire lumen and the second steering wire 132 may be disposed within the second steering wire lumen. The first steering wire 130 and the second steering wire 132 may be fixedly attached (e.g., bonded, welded, etc.) to the distal pull ring 150. For example, a distal end of the first steering wire 130 may be fixedly attached to the distal pull ring 150 and a distal end of the second steering wire 132 may be fixedly attached to the distal pull ring 150 at a position opposite the distal end of the first steering wire 130 relative to the central longitudinal axis of the elongate sheath 140. Some suitable but non-limiting materials for the first steering wire 130 and the second steering wire 132 are described below.

In some embodiments, the elongate sheath 140 may be sized in accordance with its intended use. For example, the elongate sheath 140 can have a length that is in the range of about 50 to about 200 centimeters, about 75 to about 175 centimeters, or about 100 to about 150 centimeters. Other lengths are also contemplated. It is further contemplated that the outer diameter of the elongate sheath 140 may vary based on the use or application. In some examples, the outer diameter of the elongate sheath 140 may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some embodiments, the outer diameter of the elongate sheath 140 may be a maximum of 5.66 mm (17 French), and is preferably smaller than 5.66 mm (17 French). Other configurations are also contemplated. Some suitable but non-limiting materials for the elongate sheath 140, which may include polymeric materials, metallic materials, composite materials, etc., are described below.

The first steering wire 130 may extend through the elongate sheath 140 from the axial translation mechanism 120 and/or the first carriage member 160 to the distal pull ring 150. The second steering wire 132 may extend through the elongate sheath 140 from the axial translation mechanism 120 and/or the second carriage member 170 to the distal pull ring 150. The second steering wire 132 may be disposed on an opposite side of the elongate sheath 140 from the first steering wire 130 relative to a central longitudinal axis of the elongate sheath 140.

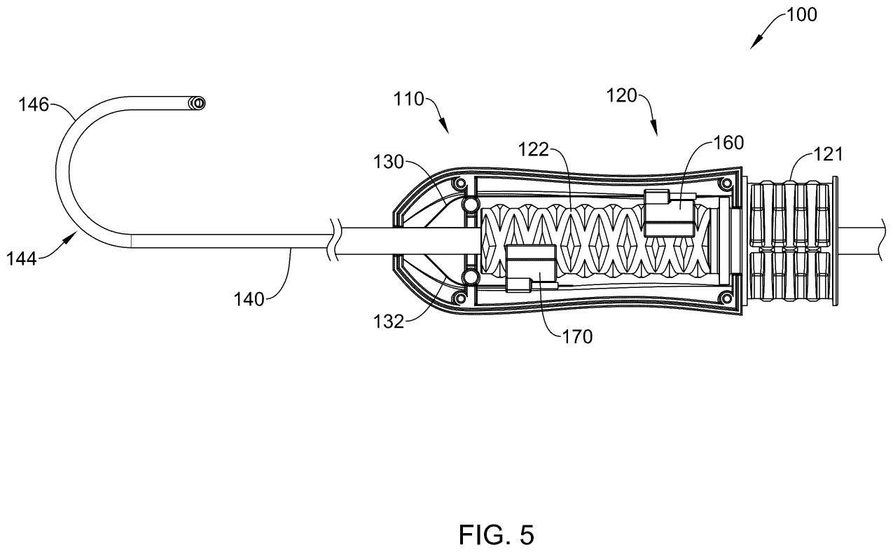

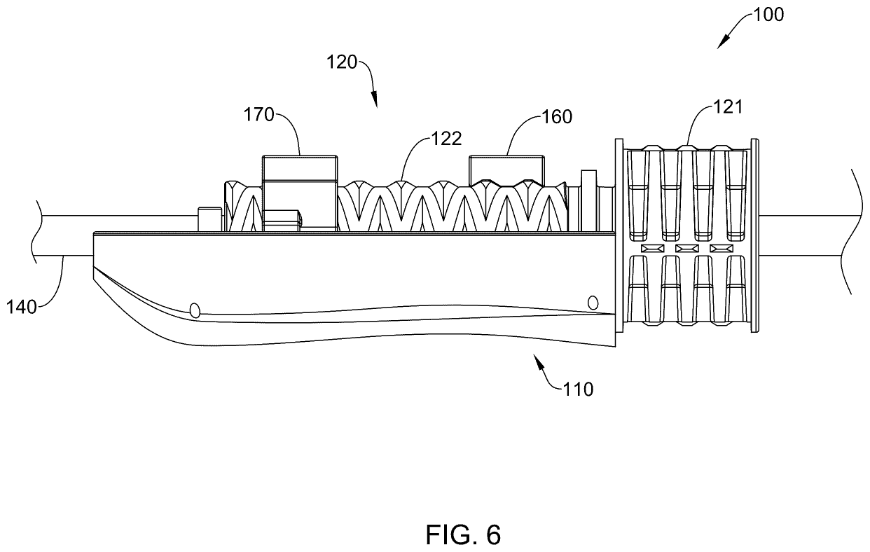

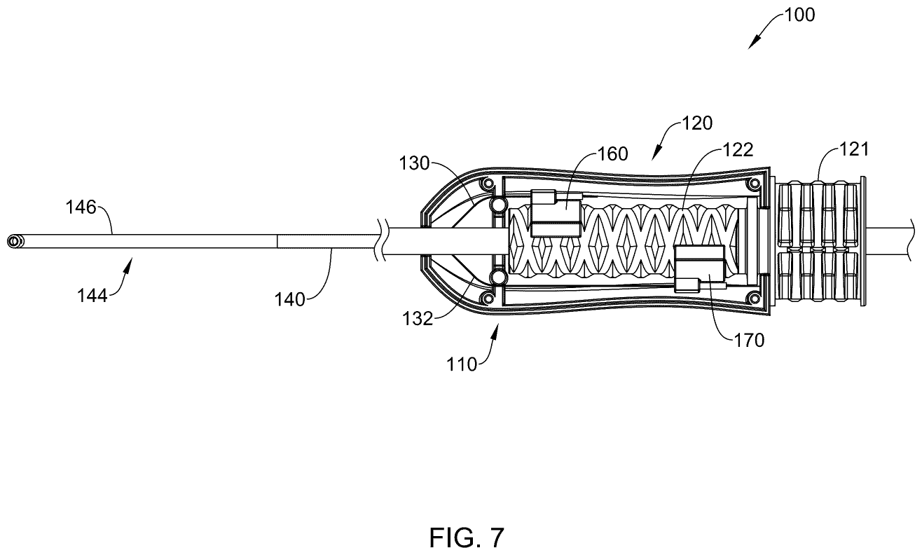

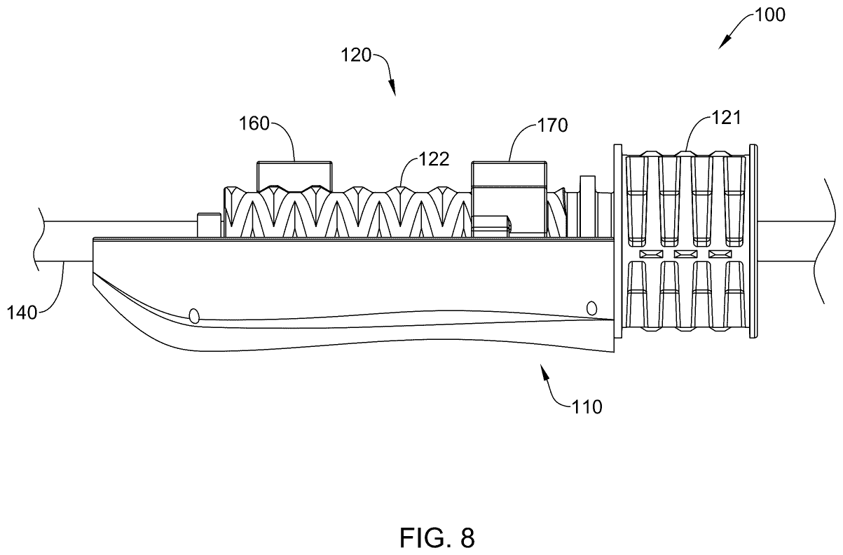

In some embodiments, the steering mechanism and/or the axial translation mechanism 120 may be configured to shift the elongate sheath 140 extending distally from the handle 110 between the relaxed configuration (e.g., FIGS. 1-2), the deflected configuration (e.g., FIGS. 5-6), and the straightened configuration (e.g., FIGS. 7-8).

In some embodiments, rotation of the rotatable knob 121 in a clockwise direction, as viewed along the bi-directional steerable catheter 100 in the proximal to distal direction, may cause clockwise rotation of the elongate shaft 122 within and/or relative to the handle 110 and/or the handle housing. In some embodiments, rotation of the rotatable knob 121 in a counterclockwise direction, as viewed along the bi-directional steerable catheter 100 in the proximal to distal direction, may cause counterclockwise rotation of the elongate shaft 122 within and/or relative to the handle 110 and/or the handle housing.

In some embodiments, rotation of the rotatable knob 121 and/or the elongate shaft 122 may be configured to actuate the axial translation mechanism 120. In some embodiments, rotation of the rotatable knob 121 and/or the elongate shaft 122 may be configured to translate the first carriage member 160 and the second carriage member 170 along the elongate shaft 122 and/or within the handle housing. In some embodiments, rotation of the rotatable knob 121 and/or the elongate shaft 122 may be configured to translate the first carriage member 160 and the second carriage member 170 in opposite directions along the elongate shaft 122 and/or within the handle housing. In some embodiments, actuation of the axial translation mechanism 120 may be configured to selectively apply tension to the first steering wire 130 and/or the second steering wire 132 to shift the elongate sheath 140 between the relaxed configuration, the deflected configuration, and the straightened configuration. In some embodiments, the first carriage member 160 and the second carriage member 170 may be configured to axially pass by each other as they translate along the elongate shaft 122 and/or within the handle housing.

Tension may be applied to the first steering wire 130 and/or the second steering wire 132 as described herein to bend and/or deflect the distal portion 144 and/or the first curve 146 of the elongate sheath 140. The first steering wire 130 may be configured to engage the axial translation mechanism 120 to bend and/or deflect the distal portion 144 and/or the first curve 146 of the elongate sheath 140 in the first direction toward the handle 110 and/or the handle housing, toward and/or to the deflected configuration (e.g., FIG. 1). The second steering wire 132 may be configured to engage the axial translation mechanism 120 to bend and/or deflect the distal portion 144 and/or the first curve 146 of the elongate sheath 140 in the second direction opposite the first direction and away from the handle 110 and/or the handle housing, toward and/or to the straightened configuration (e.g., FIG. 1). The tension applied by the axial translation mechanism 120 may be sufficient to overcome the self-bias of the elongate sheath 140 toward the normal or relaxed configuration (where present) and bend and/or deflect the distal portion 144 and/or the first curve 146 of the elongate sheath 140 in the first direction and/or the second direction.

In some embodiments, the threaded portion (e.g., the first helical thread 123, the second helical thread 124) of the elongate shaft 122 may be configured to provide self-locking threads and/or a self-locking function. Self-locking threads require a torque input to facilitate relative movement between elements of the axial translation mechanism 120, and are configured to “lock” in position when the torque input is removed. For example, rotation of the elongate shaft 122 in the clockwise direction or the counterclockwise direction may cause actuation of the axial translation mechanism 120 as described herein. However, when the rotational force is removed, the axial translation mechanism 120 may remain fixed in its last position, thereby preventing actuation and/or relative movement of the axial translation mechanism 120. In order to facilitate the self-locking function of the threaded portion (e.g., the first helical thread 123, the second helical thread 124) of the elongate shaft 122, design parameters of the threads are carefully selected to meet the desired design intent. FIG. 9A, which illustrates a portion of the elongate shaft 122 shown in FIG. 9 in greater detail, illustrates several of the design parameters that may be selected to provide the desired self-locking function. The design parameters themselves shall be understood to be applicable to both the first helical thread 123 and the second helical thread 124, even if not explicitly discussed in the context of each.

In order to have self-locking threads and/or the self-locking function, the coefficient of friction f (unitless) between the material of the elongate shaft 122 and the material(s) of the first carriage member 160 and the second carriage member 170 must be greater than or equal to the equation below:

$$f > = ( L * cos An ) / ( π * Dm )$$

In FIG. 9A and the equation above:
P refers to the PITCH of the threads.
L refers to the LEAD of the threads, where L=P*(number of thread starts).
An refers to the THREAD ANGLE in the normal plane.
Dm refers to the MEAN DIAMETER OF THREAD CONTACT.

References P, L, and Dm are shown in FIG. 9A. Additionally, in FIG. 9A, the thread angle A (in the axial plane) is illustrated. In order to convert from the thread angle A (in the axial plane) to the thread angle An (in the normal plane), the following equation is used:

$$tan An = tan A * cos H$$

### Where:

A refers to the THREAD ANGLE in the axial plane, shown in FIG. 9A.
H refers to the HELIX ANGLE, shown in FIG. 9A.

The above equation for f was used as a guide to identify design parameters that may be varied to produce self-locking threads and/or the self-locking function. It should be noted that the above equation for f was developed for and is applicable to a single helix thread. In the current example, the threaded portion of the elongate shaft 122 includes a double start thread and a double helix thread. As such, experimentation was required to find parameters and values that produce the desired result.

Based on the above equation for the coefficient of friction f, it is believed that in order to provide self-locking threads and/or the self-locking function, the coefficient of friction f should be between 0.1 and 0.2 in air (e.g., without the aid of a secondary lubricating agent, discussed further below). In one example, such as with interacting components having a combination of materials including POM-H and ABS, in order to provide self-locking threads and/or the self-locking function, the coefficient of friction f should be greater than 0.13 in air. Depending on particular materials used, other values for the coefficient of friction f may produce the desired self-locking function. As such, other material combinations may be used and/or other values for the design parameters may be used, either alone or in combination, to produce the desired self-locking function.

Providing smooth actuation of the axial translation mechanism 120 while simultaneously providing self-locking function is also desirable and requires a delicate balance of design parameters including, but not necessarily limited to, the lead L, the thread angle A, and/or the mean diameter Dm.

In some embodiments, for the double-start example disclosed herein, the lead L may be between 0.245 inches (in) (6.223 millimeters (mm)) and 0.585 in (14.859 mm). In some embodiments, the lead L may be between 0.28 in (7.112 mm) and 0.54 in (13.716 mm). In some embodiments, the lead L may be between 0.315 in (8.001 mm) and 0.495 in (12.573 mm). In one preferred but non-limiting example, the lead L may be between 0.35 in (8.89 mm) and 0.45 in (11.43 mm).

In some embodiments, for the double-start example disclosed herein, the thread angle A may be between 35 degrees and 78 degrees. In some embodiments, the thread angle A may be between 40 degrees and 72 degrees. In some embodiments, the thread angle A may be between 45 degrees and 66 degrees. In one preferred but non-limiting example, the thread angle A may be between 50 degrees and 60 degrees.

In some embodiments, for the double-start example disclosed herein, the mean diameter of thread contact Dm may be between 0.35 in (8.89 mm) and 0.91 in (23.114 mm). In some embodiments, the mean diameter of thread contact Dm may be between 0.40 in (10.16 mm) and 0.84 in (21.336 mm). In some embodiments, the mean diameter of thread contact Dm may be between 0.45 in (11.43 mm) and 0.77 in (19.558 mm). In one preferred but non-limiting example, the mean diameter of thread contact Dm may be between 0.50 in (12.7 mm) and 0.70 in (17.78 mm).

In at least some embodiments, the axial translation mechanism 120 is devoid of a secondary lubricating agent (e.g., oil, grease, etc.). For the purpose of this disclosure, a secondary lubricating agent does not include materials, coatings, etc. that are bonded, fixedly attached, etc. to the components and/or elements of the axial translation mechanism 120. As such, the materials for the elongate shaft 122, the first carriage member 160, and the second carriage member 170 may be selected to facilitate relative movement therebetween while considering a handheld “power grip” per human factors standards (e.g., to minimize torque required to rotate the elongate shaft 122 to actuate the axial translation mechanism 120) while maintaining the self-locking function when the elongate shaft 122 is not being rotated. Secondary lubricating agents are sometimes used when trying to minimize torque application. However, the use of a secondary lubricating agent may increase the risk of contamination, adds cost and steps during manufacturing and/or assembly, and may be messy. As such, it is desirable to avoid the use of a secondary lubricating agent.

FIGS. 5 and 6 are views illustrating actuation of the axial translation mechanism 120 and/or axial translation of the first carriage member 160 and the second carriage member 170 corresponding to shifting the elongate sheath 140 toward and/or to the deflected configuration. It should also be noted that there is at least one change of scale in FIG. 5. In some embodiments, clockwise rotation of the elongate shaft 122 may be configured to and/or may cause proximal movement and/or proximal axial translation of the first carriage member 160. Proximal movement and/or proximal axial translation of the first carriage member 160 may apply tension to the first steering wire 130, thereby shifting the elongate sheath toward and/or to the deflected configuration. In some embodiments, clockwise rotation of the elongate shaft 122 may be configured to and/or may cause distal movement and/or distal axial translation of the second carriage member 170. Distal movement and/or distal axial translation of the second carriage member 170 may release stored tension within the second steering wire 132 and/or may permit the second steering wire 132 to translate distally within the elongate sheath 140 such that the distal pull ring 150 may be pulled by the first steering wire 130.

FIGS. 7 and 8 are views illustrating actuation of the axial translation mechanism 120 and/or axial translation of the first carriage member 160 and the second carriage member 170 corresponding to shifting the elongate sheath 140 toward and/or to the straightened configuration. It should also be noted that there is at least one change of scale in FIG. 7. In some embodiments, counterclockwise rotation of the elongate shaft 122 may be configured to and/or may cause proximal movement and/or proximal axial translation of the second carriage member 170. Proximal movement and/or proximal axial translation of the second carriage member 170 may apply tension to the second steering wire 132, thereby shifting the elongate sheath toward and/or to the straightened configuration. In some embodiments, clockwise rotation of the elongate shaft 122 may be configured to and/or may cause distal movement and/or distal axial translation of the first carriage member 160. Distal movement and/or distal axial translation of the first carriage member 160 may release stored tension within the first steering wire 130 and/or may permit the first steering wire 130 to translate distally within the elongate sheath 140 such that the distal pull ring 150 may be pulled by the second steering wire 132.

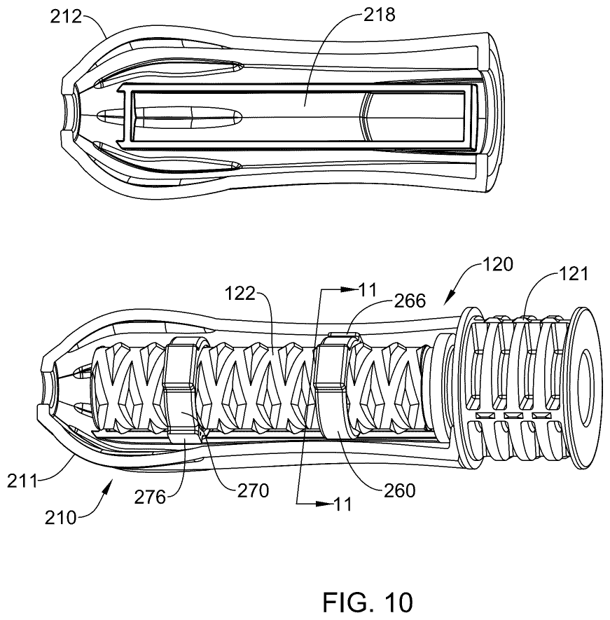

Turning now to FIGS. 10-13, selected aspects of an alternative configuration of a handle 210 usable with the bi-directional steerable catheter 100 are illustrated. In the interest of clarity, some elements and/or features are not shown. Except where noted, features and/or elements discussed above with respect to the handle 110 and/or the bi-directional steerable catheter 100 may also be used with the handle 210. For example, the elongate sheath 140 as described herein may extend distally from the handle 210.

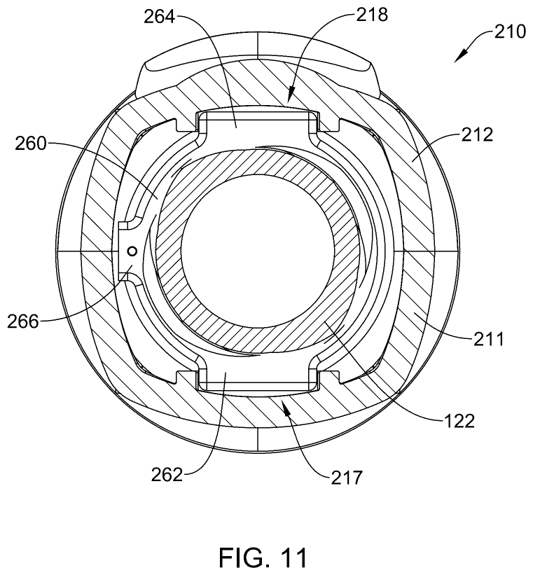

The handle 210 may include a handle housing. The handle housing may comprise a first handle shell 211 (e.g., a right handle shell, a bottom handle shell, etc.) and a second handle shell 212 (e.g., a left handle shell, a top handle shell, etc.). In some embodiments, the handle housing may form a clam shell configuration. In some embodiments, the first handle shell 211 and the second handle shell 212 may be secured together, using fasteners, a snap fit, an interference fit, etc. Some suitable but non-limiting materials for the handle 210, the handle housing, the first handle shell 211, the second handle shell 212, etc., including but not limited to polymeric materials, metallic materials, and/or composite materials, are described below. In some embodiments, the handle 210, the handle housing, the first handle shell 211, the second handle shell 212 may preferably be formed from a polymeric material. In one non-limiting example, the handle 210, the handle housing, the first handle shell 211, the second handle shell 212 may preferably be formed from acrylonitrile butadiene styrene (ABS). Other configurations and/or materials are also contemplated. In the view shown in FIG. 10, the second handle shell 212 of the handle housing has been flipped over to show some internal components of the handle 210. The cross-sectional view of FIG. 11 is shown with the second handle shell 212 of the handle housing in place (e.g., as seen in FIG. 1) to illustrate relative positioning of and/or interaction between selected features.

In some embodiments, the bi-directional steerable catheter 100 may comprise a steering mechanism comprising an axial translation mechanism 120 disposable within and/or disposed within the handle 210 of the bi-directional steerable catheter 100. In some embodiments, the axial translation mechanism 120 may comprise an elongate shaft 122 having a circumference. In some embodiments, the elongate shaft 122 may have a threaded portion. In some embodiments, the elongate shaft 122 may be rotatably disposable within and/or rotatably disposed within the handle housing. In some embodiments, the elongate shaft 122 may be rotatable relative to the handle housing.

In some embodiments, the axial translation mechanism 120 may comprise a first carriage member 260 couplable to the first steering wire 130 (not shown). In some embodiments, the steering mechanism may comprise the axial translation mechanism 120 coupled to the first steering wire 130. In some embodiments, the first carriage member 260 may be coupled to the first steering wire 130. In some embodiments, the first carriage member 260 may be operatively engaged with the elongate shaft 122 and/or the threaded portion of the elongate shaft 122. In some embodiments, the first carriage member 260 may be operatively engaged with the first helical thread 123 (e.g., FIG. 9) and/or the clockwise discontinuous threading. In some embodiments, the first steering wire 130 may be configured to extend and/or may extend within and/or through the elongate sheath 140.

In some embodiments, the axial translation mechanism 120 may comprise a second carriage member 270 couplable to the second steering wire 132 (not shown). In some embodiments, the steering mechanism may comprise the axial translation mechanism 120 coupled to the second steering wire 132. In some embodiments, the second carriage member 270 may be coupled to the second steering wire 132. In some embodiments, the second carriage member 270 may be operatively engaged with the elongate shaft 122 and/or the threaded portion of the elongate shaft 122. In some embodiments, the second carriage member 270 may be operatively engaged with the second helical thread 124 (e.g., FIG. 9) and/or the counterclockwise discontinuous threading. In some embodiments, the second steering wire 132 may be configured to extend and/or may extend within and/or through the elongate sheath 140.

In some embodiments, the handle housing may comprise one or more slide features configured to guide and/or constrain the first carriage member 260 and the second carriage member 270. In some embodiments, the one or more slide features may permit axial sliding movement of the first carriage member 260 and the second carriage member 270 within the handle housing. In some embodiments, the one or more slide features may limit and/or constrain the first carriage member 260 and the second carriage member 270 to axial sliding movement within the handle housing. In some embodiments, the first handle shell 211 may comprise a first guide slot 217 extending longitudinally along an inner surface of the first handle shell 211. The first guide slot 217 of the first handle shell 211 may be configured to engage with and/or receive the first carriage member 260 and the second carriage member 270. In some embodiments, the second handle shell 212 may comprise a second guide slot 218 extending longitudinally along an inner surface of the second handle shell 212. The second guide slot 218 of the second handle shell 212 may be configured to engage with and/or receive the first carriage member 260 and the second carriage member 270, as seen in FIG. 11, which is a cross-sectional view taken along the line 11-11 in FIG. 10. In some embodiments, the first guide slot 217 of the first handle shell 211 may be disposed opposite the second guide slot 218 of the second handle shell 212 with respect to a plane containing a central longitudinal axis of the handle 210 and/or a central longitudinal axis of the elongate shaft 122.

In some embodiments, the first carriage member 260 may comprise a first slide extension 262 and a second slide extension 264, as seen in FIG. 11. The first slide extension 262 of the first carriage member 260 may be configured to slidably engage and/or may be slidably engaged with the first guide slot 217 of the first handle shell 211. The second slide extension 264 of the first carriage member 260 may be configured to slidably engage and/or may be slidably engaged with the second guide slot 218 of the second handle shell 212. In some embodiments, the first carriage member 260 may extend circumferentially around the circumference of the elongate shaft 122. In some embodiments, the first carriage member 260 may extend completely around the circumference of the elongate shaft 122. In some embodiments, the first carriage member 260 may comprise a first ear 266 extending radially outward from the elongate shaft 122 and/or the first carriage member 260. The first ear 266 may be configured to receive and/or engage with the first steering wire 130. In some embodiments, the first steering wire 130 may pass through an aperture formed in the first ear 266. In some embodiments, the first steering wire 130 may comprise a first wire lug (not shown) disposed proximal of the first ear 266, wherein the first wire lug is configured to engage with the first ear 266 and is unable to pass through the aperture formed in the first ear 266. In some embodiments, the first wire lug may be configured to couple the first steering wire 130 to the first carriage member 260 and/or the first ear 266.

In some embodiments, the second carriage member 270 may comprise a first slide extension (not shown) and a second slide extension (not shown). The first slide extension of the second carriage member 270 may be configured to slidably engage and/or may be slidably engaged with the first guide slot 217 of the first handle shell 211 similar to the first slide extension 262 of the first carriage member 260 above. The second slide extension of the second carriage member 270 may be configured to slidably engage and/or may be slidably engaged with the second guide slot 218 of the second handle shell 212 similar to the second slide extension 264 of the first carriage member 260 above. In some embodiments, the second carriage member 270 may extend circumferentially around the circumference of the elongate shaft 122. In some embodiments, the second carriage member 270 may extend completely around the circumference of the elongate shaft 122. In some embodiments, the second carriage member 270 may comprise a second ear 276 extending radially outward from the elongate shaft 122 and/or the second carriage member 270. The second ear 276 may be configured to receive and/or engage with the second steering wire 132. In some embodiments, the second steering wire 132 may pass through an aperture formed in the second ear 276. In some embodiments, the second steering wire 132 may comprise a second wire lug (not shown) disposed proximal of the second ear 276, wherein the second wire lug is configured to engage with the second ear 276 and is unable to pass through the aperture formed in the second ear 276. In some embodiments, the second wire lug may be configured to couple the second steering wire 132 to the second carriage member 270 and/or the second ear 276.

In some embodiments, the first carriage member 260 and the second carriage member 270 may be disposed coaxial with the elongate shaft 122 and/or the central longitudinal axis of the elongate shaft 122. In some embodiments, the first carriage member 260 and/or the second carriage member 270 may be formed from a polymeric material. In one non-limiting example, the first carriage member 260 and/or the second carriage member 270 may be formed from polyoxymethylene homopolymer (POM-H; for example, DELRIN®). Some other examples of polymeric materials that may be suitable for use with the first carriage member 260 and/or the second carriage member 270 are discussed below.

In some embodiments, rotation of the rotatable knob 121 in a clockwise direction, as viewed along the bi-directional steerable catheter 100 in the proximal to distal direction, may cause clockwise rotation of the elongate shaft 122 within and/or relative to the handle 210 and/or the handle housing. In some embodiments, rotation of the rotatable knob 121 in a counterclockwise direction, as viewed along the bi-directional steerable catheter 100 in the proximal to distal direction, may cause counterclockwise rotation of the elongate shaft 122 within and/or relative to the handle 210 and/or the handle housing.

In some embodiments, rotation of the rotatable knob 121 and/or the elongate shaft 122 may be configured to actuate the axial translation mechanism 120. In some embodiments, rotation of the rotatable knob 121 and/or the elongate shaft 122 may be configured to translate the first carriage member 260 and the second carriage member 270 along the elongate shaft 122 and/or within the handle housing. In some embodiments, rotation of the rotatable knob 121 and/or the elongate shaft 122 may be configured to translate the first carriage member 260 and the second carriage member 270 in opposite directions along the elongate shaft 122 and/or within the handle housing. In some embodiments, actuation of the axial translation mechanism 120 may be configured to selectively apply tension to the first steering wire 130 and/or the second steering wire 132 to shift the elongate sheath 140 between the relaxed configuration, the deflected configuration, and the straightened configuration.

Tension may be applied to the first steering wire 130 and/or the second steering wire 132 as described herein to bend and/or deflect the distal portion 144 and/or the first curve 146 of the elongate sheath 140. The first steering wire 130 may be configured to engage the axial translation mechanism 120 to bend and/or deflect the distal portion 144 and/or the first curve 146 of the elongate sheath 140 in the first direction toward the handle 210 and/or the handle housing, toward and/or to the deflected configuration. The second steering wire 132 may be configured to engage the axial translation mechanism 120 to bend and/or deflect the distal portion 144 and/or the first curve 146 of the elongate sheath 140 in the second direction opposite the first direction and away from the handle 210 and/or the handle housing, toward and/or to the straightened configuration. The tension applied by the axial translation mechanism 120 may be sufficient to overcome the self-bias of the elongate sheath 140 toward the normal or relaxed configuration (where present) and bend and/or deflect the distal portion 144 and/or the first curve 146 of the elongate sheath 140 in the first direction and/or the second direction.

Similar to above, in at least some embodiments, the axial translation mechanism 120 is devoid of a secondary lubricating agent (e.g., oil, grease, etc.). In some embodiments, the materials for the elongate shaft 122, the first carriage member 260, and the second carriage member 270 may be selected to facilitate relative movement therebetween while considering a handheld “power grip” per human factors standards (e.g., to minimize torque required to rotate the elongate shaft 122 to actuate the axial translation mechanism 120) while maintaining the self-locking function when the elongate shaft 122 is not being rotated. Secondary lubricating agents are sometimes used when trying to minimize torque application. However, the use of a secondary lubricating agent may increase the risk of contamination, adds cost and steps during manufacturing and/or assembly, and may be messy. As such, it is desirable to avoid the use of a secondary lubricating agent.

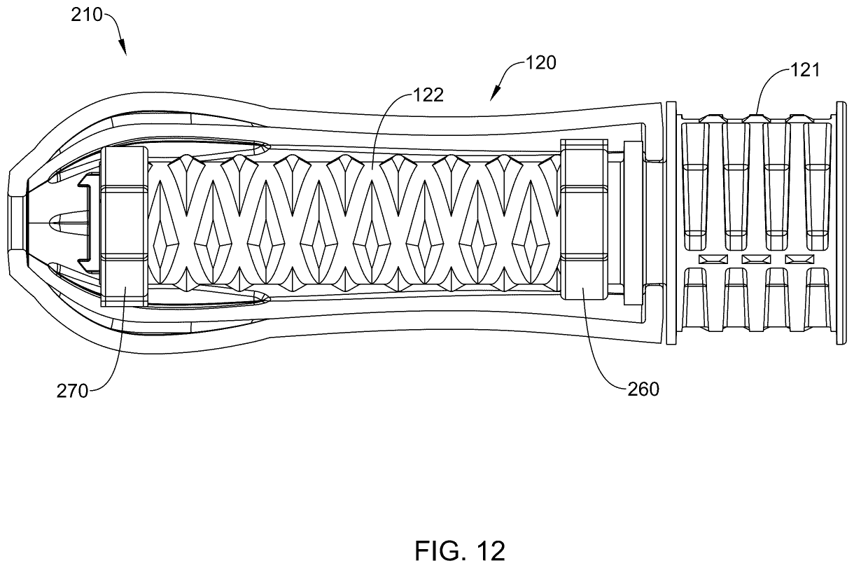

FIG. 12 is a side view illustrating actuation of the axial translation mechanism 120 and/or axial translation of the first carriage member 260 and the second carriage member 270 corresponding to shifting the elongate sheath 140 toward and/or to the deflected configuration. In some embodiments, clockwise rotation of the elongate shaft 122 may be configured to and/or may cause proximal movement and/or proximal axial translation of the first carriage member 260. Proximal movement and/or proximal axial translation of the first carriage member 260 may apply tension to the first steering wire 130 (not shown), thereby shifting the elongate sheath toward and/or to the deflected configuration. In some embodiments, clockwise rotation of the elongate shaft 122 may be configured to and/or may cause distal movement and/or distal axial translation of the second carriage member 270. Distal movement and/or distal axial translation of the second carriage member 270 may release stored tension within the second steering wire 132 (not shown) and/or may permit the second steering wire 132 to translate distally within the elongate sheath 140 such that the distal pull ring 150 may be pulled by the first steering wire 130. In some embodiments, the first carriage member 260 and the second carriage member 270 may be configured to axially translate apart from each other as the elongate sheath 140 is shifted toward and/or to the deflected configuration.

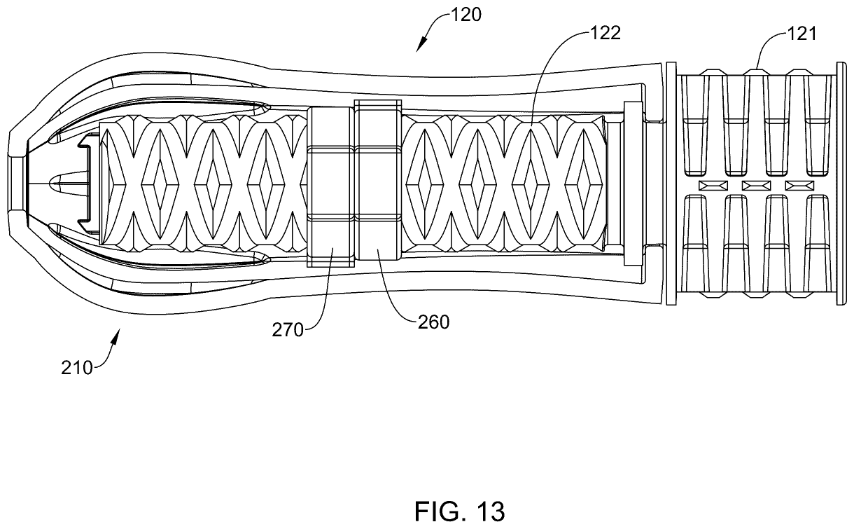

FIG. 13 is a side view illustrating actuation of the axial translation mechanism 120 and/or axial translation of the first carriage member 260 and the second carriage member 270 corresponding to shifting the elongate sheath 140 toward and/or to the straightened configuration. In some embodiments, counterclockwise rotation of the elongate shaft 122 may be configured to and/or may cause proximal movement and/or proximal axial translation of the second carriage member 270. Proximal movement and/or proximal axial translation of the second carriage member 270 may apply tension to the second steering wire 132, thereby shifting the elongate sheath toward and/or to the straightened configuration. In some embodiments, clockwise rotation of the elongate shaft 122 may be configured to and/or may cause distal movement and/or distal axial translation of the first carriage member 260. Distal movement and/or distal axial translation of the first carriage member 260 may release stored tension within the first steering wire 130 and/or may permit the first steering wire 130 to translate distally within the elongate sheath 140 such that the distal pull ring 150 may be pulled by the second steering wire 132. In some embodiments, the first carriage member 260 and the second carriage member 270 may be configured to axially translate toward each other as the elongate sheath 140 is shifted toward and/or to the straightened configuration. In some embodiments, the first carriage member 260 and the second carriage member 270 may be in contact with each other when the elongate sheath 140 is disposed in the straightened configuration.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the handle, the elongate sheath, the steering mechanism, the axial translation mechanism, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-clastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antincoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment in other embodiments. The scope of the disclosure is, of course, defined in the language in which the appended claims are expressed.

What is claimed is:

1. A steering mechanism for a bi-directional steerable catheter, comprising:
an axial translation mechanism disposable within a handle of the bi-directional steerable catheter, wherein the axial translation mechanism comprises:
an elongate shaft having a circumference and comprising a first helical thread extending along a length of the elongate shaft in a first helical direction, and a second helical thread extending along the length of the elongate shaft in a second helical direction different from the first helical direction;
a first carriage member couplable to a first steering wire and operatively engaged with the first helical thread; and
a second carriage member couplable to a second steering wire and operatively engaged with the second helical thread;
wherein rotation of the elongate shaft is configured to translate the first carriage member and the second carriage member in opposite directions along the elongate shaft.

2. The steering mechanism of claim 1, wherein the first carriage member extends circumferentially around at least a portion of the circumference of the elongate shaft.

3. The steering mechanism of claim 1, wherein the second carriage member extends circumferentially around at least a portion of the circumference of the elongate shaft.

4. The steering mechanism of claim 1, wherein the first carriage member and the second carriage member are configured to axially pass by each other as they translate along the elongate shaft.

5. The steering mechanism of claim 1, wherein the first carriage member and the second carriage member are disposed coaxial with the elongate shaft.

6. The steering mechanism of claim 1, further comprising a knob configured to rotate relative to the handle, wherein the knob is fixedly attached to the elongate shaft.

7. The steering mechanism of claim 6, wherein the knob is axially spaced apart from the first helical thread and the second helical thread.

8. The steering mechanism of claim 1, wherein the axial translation mechanism is configured to shift an elongate sheath extending distally from the handle between a relaxed configuration, a deflected configuration, and a straightened configuration.

9. The steering mechanism of claim 8, further comprising:
a first steering wire configured to extend through the elongate sheath from the first carriage member to a distal pull ring disposed adjacent a distal tip of the elongate sheath; and
a second steering wire configured to extend through the elongate sheath from the second carriage member to the distal pull ring;
wherein actuation of the axial translation mechanism is configured to selectively apply tension to the first steering wire and the second steering wire to shift the elongate sheath between the relaxed configuration, the deflected configuration, and the straightened configuration.

10. A bi-directional steerable catheter, comprising:
a handle comprising a steering mechanism; and
an elongate sheath extending distally from the handle;
wherein the steering mechanism comprises an axial translation mechanism disposed within the handle, the axial translation mechanism being coupled to a first steering wire extending within the elongate sheath and a second steering wire extending within the elongate sheath;
wherein the axial translation mechanism comprises:
an elongate shaft having a circumference and comprising a first helical thread extending along an exterior of the elongate shaft in a first helical direction, and a second helical thread extending along the exterior of the elongate shaft in a second helical direction different from the first helical direction, wherein the first helical thread axially overlaps the second helical thread;
a first carriage member operatively engaged with the first helical thread, the first carriage member being coupled to the first steering wire; and
a second carriage member operatively engaged with the second helical thread, the second carriage member being coupled to the second steering wire;
wherein rotation of the elongate shaft is configured to translate the first carriage member and the second carriage member in opposite directions along the elongate shaft.

11. The bi-directional steerable catheter of claim 10, wherein the first helical thread intersects the second helical thread.

12. The bi-directional steerable catheter of claim 10, wherein the first helical thread is discontinuous along the elongate shaft.

13. The bi-directional steerable catheter of claim 10, wherein the second helical thread is discontinuous along the elongate shaft.

14. The bi-directional steerable catheter of claim 10, further comprising a rotatable knob disposed outside of the handle, wherein the rotatable knob is fixedly attached to the elongate shaft.

15. The bi-directional steerable catheter of claim 14, wherein the rotatable knob is monolithically formed with the elongate shaft.

16. A bi-directional steerable catheter, comprising:
a handle comprising a steering mechanism; and
an elongate sheath extending distally from the handle;
wherein the steering mechanism comprises an axial translation mechanism coupled to a first steering wire extending within the elongate sheath and a second steering wire extending within the elongate sheath, the axial translation mechanism comprising:
an elongate shaft having a threaded portion comprising clockwise discontinuous threading overlaid with counterclockwise discontinuous threading;
a first carriage member operatively engaged with the threaded portion, the first carriage member being coupled to the first steering wire; and
a second carriage member operatively engaged with the threaded portion, the second carriage member being coupled to the second steering wire;
wherein rotation of the elongate shaft is configured to translate the first carriage member and the second carriage member in opposite directions along the elongate shaft.

17. The bi-directional steerable catheter of claim 16, wherein the clockwise discontinuous threading and the counterclockwise discontinuous threading are formed by intersecting helical grooves extending radially inward from an outermost extent of the threaded portion.

18. The bi-directional steerable catheter of claim 17, wherein the intersecting helical grooves form a plurality of diamond-shaped projections extending radially outward from an innermost extent of the intersecting helical grooves.

19. The bi-directional steerable catheter of claim 18, wherein each diamond-shaped projection of the plurality of diamond-shaped projections forms a portion of the clockwise discontinuous threading and a portion of the counterclockwise discontinuous threading.

20. The bi-directional steerable catheter of claim 16, wherein the first carriage member is operatively engaged with the clockwise discontinuous threading and the second carriage member is operatively engaged with the counterclockwise discontinuous threading.
